(19) United States
(12) Patent Application Publication
(10) Pub. No.: US 2019/0054044 A1
(43) Pub. Date: Feb. 21, 2019

(54) ORAL DOSAGE FORM OF BOTH CLOMIPHENE ISOMERS AND METHOD OF USING SAME TO TREAT SECONDARY HYPOGONADISM AND MINIMIZE DRUG SIDE EFFECTS IN MEN

(71) Applicant: ASPEN PARK PHARMACEUTICALS, INC., Scarsdale, NY (US)

(72) Inventors: Mitchell Steiner, Germantown, TN (US); Harry Fisch, Scarsdale, NY (US)

(73) Assignee: ASPEN PARK PHARMACEUTICALS, INC., Scarsdale, NY (US)

(21) Appl. No.: 16/079,623

(22) PCT Filed: Feb. 23, 2017
(86) PCT No.: PCT/US17/19081

§ 371 Date: Aug. 24, 2018

Related U.S. Application Data
(60) Provisional application No. 62/299,686, filed on Feb. 25, 2016.

(51) Int. Cl. A61K 31/138; A61K 9/20; A61P 5/26

(52) U.S. Cl. CPC A61K 31/138; A61P 5/26; A61K 9/2018; A61K 9/2059

(57) ABSTRACT

The present invention relates to a pharmaceutical oral dosage form comprising both (A) trans-clomiphene or a pharmaceutically acceptable salt thereof and (B) cis-clomiphene or a pharmaceutically acceptable salt thereof, wherein (i) the ratio of (A):(B) in the dosage form is about 70:30; (ii) the amount of (A) in the dosage form is about 12.5 mg or about 25 mg; and (iii) the amount of (B) in the dosage form is less than 15 mg. The pharmaceutical oral dosage form is useful to treat secondary hypogonadism in men and minimize certain antiestrogenic drug side effects such as impaired cognition, hot flashes and bone loss, osteoporosis, and skeletal fractures.

### BACKGROUND OF THE INVENTION

### 1. Field of the Invention

The present invention relates to a pharmaceutical oral dosage form containing effective quantities of each of the two clomiphene isomers (cis- and trans-geometric isomers) or a pharmaceutically acceptable salt thereof and the use of the pharmaceutical oral dosage form to treat secondary hypogonadism and minimize drug side effects in men.

### 2. Description of Related Art

Testosterone (T) is a primary androgenic hormone produced in the interstitial cells of the testes and is responsible for normal growth, development and maintenance of male sex organs and secondary sex characteristics (e.g., deepening voice, muscular development, facial hair, etc.). Throughout adult life, testosterone is necessary for proper functioning of the testes and its accessory structures, prostate and seminal vesicle; for sense of well-being; and for maintenance of libido, erectile potency.

Testosterone deficiency—insufficient secretion of T characterized by low serum T concentrations—can give rise to hypogonadism in males. The hypogonadism is characterized as “primary” if the cause is testicular failure. The hypogonadism is characterized as “secondary” if the cause is inadequate secretion of the pituitary gonadotropins. There are instances where the patient suffers a mixture of both primary and secondary hypogonadism. Symptoms associated with male hypogonadism include impotence and decreased sexual desire, fatigue and loss of energy, mood depression, regression of secondary sexual characteristics, decreased muscle mass, and increased fat mass. Furthermore, hypogonadism in men is a risk factor for osteoporosis, metabolic syndrome, type II diabetes and cardiovascular disease.

Various testosterone replacement therapies are commercially available for the treatment of male hypogonadism. Pharmaceutical preparations include both testosterone and testosterone derivatives in the form of intramuscular injections, implants, oral tablets of alkylated T (e.g., methyltestosterone), topical gels, or topical patches. All of the current T therapies, however, fail to adequately provide an easy and clinically effective method of delivering T. For example, intramuscular injections are painful and are associated with significant fluctuations in serum T levels between doses; T patches are generally associated with levels of T in the lower range of normal (i.e., clinically ineffective) and often cause substantial skin irritation; and T gels have been associated with unsafe transfer of T from the user to women and children. In addition, men undergoing T replacement therapies are at risk of enlarged breasts, shrunken testicles, water retention, acne, sleep apnea and baldness. The only oral testosterone product ever approved to date by the FDA is methyltestosterone (in which a methyl group covalently bound to a testosterone “nucleus” at the C-17 position to inhibit hepatic metabolism; note, also, that methyltestosterone is not a prodrug of testosterone) and this approval occurred several decades ago. Unfortunately, use of methyltestosterone has been associated with a significant incidence of liver toxicity, and it is rarely prescribed to treat men with low testosterone. Over time, therefore, the current methods of treating testosterone deficiency suffer from poor compliance, inadequate or erratic pharmacokinetics, and significant side effects including suppression of endogenous gonadotropins (LH and FSH), T, and spermatogenesis and thus unsatisfactory treatment of men with low T because of secondary hypogonadism.

For these reasons, among others, no oral formulation of testosterone or testosterone derivatives has been approved by the United States Food and Drug Administration (FDA) to date. Hence, there remains a need for an oral formulation that delivers testosterone directly to or induces endogenous testosterone production by Leydig cells in men and provides optimum serum testosterone levels that are clinically effective to treat hypogonadal men (i.e., those with a serum T concentration of ≤300 ng/dL coupled with at least one attendant symptom) over an extended period of time.

U.S. Pat. No. 6,391,920 (Harry Fisch) identifies CLOMID® (clomiphene citrate) as an example of an antiestrogen suitable to treat secondary hypogonadism in men. CLOMID® is a mixture of two geometric isomers: (1) a trans-isomer, which is also known as enclomiphene and (2) a cis-isomer, which is also known as zuclomiphene. CLOMID® is marketed in 50 mg tablets, which, according to the U.S. Pharmacopeia, may contain anywhere from 50-70% trans isomer and, correspondingly, 50-30% cis isomer. Typically, the percent ratio of trans:cis isomer in the commercial formulation is actually 62:38%.

Although more often the trans-isomer is antiestrogenic and the cis-isomer is estrogenic on target tissues, either of these isomers may, in fact, be either estrogenic or antiestrogenic depending on concentration, relative concentrations of active and inactive clomiphene metabolites generated, pharmacokinetics (different half lives of each isomer), specific target tissue, and different affects, agonist or antagonist, on estrogen receptor types (ERα and ERβ). See, A. Kumar et al., J. Biosci., 20(5): 665-673 (1995); R. Young et al., Int. J. Fertil., 36(5): 291-5 (1991); P. Ruenitz, Biochem. Pharmacol., 32(19): 2941-7 (1983); and S. Goldstein et al, Human Reproduct. Update, 6(3): 212-224 (2000). Furthermore, the differential effects and opposing effects of the cis- and trans-isomers on pituitary and hypothalamus function also plays a role on the net estrogenic or antiestrogenic clinical effects of each or both of the clomiphene isomers. See, T. Kurosawa et al, Endocrin. J., 57(6): 517-512 (2010); J. Clark et al, Biol. Reprod., 25: 667-672 (1981); S. Hill et al, IDrugs, 12(2): 109-119 (2009); S. Goldstein et al, Human Reproduct. Update, 6(3): 212-224 (2000); and R. Wiehle et al, BJU Int., 112: 1188-1200 (2013). Consequently, generalizations cannot possibly be made to predict what the actual clinical effects of each or ratio of both of the clomiphene isomers and the actual dose of each or ratio of both of the clomiphene isomers will be. Because of all these confounding pharmacologic variables of a combination cis- and trans-clomiphene tablet, the actual ratios and doses need to be empirically ascertained depending on the desired clinical therapeutic effects to be achieved. In the case for treating secondary hypogonadism while minimizing certain unwanted drug side effects, the actual proportion of the isomers and their individual doses are not obvious and require empirical testing to achieve the intended optimal pharmacologic effects that increase pituitary secretion of LH and thereby effectively increase T levels as well as minimize clinically unwanted effects, such as bone loss and osteoporosis and hot flashes.

The '920 patent teaches administration of a dose of 10-25 mg of CLOMID® daily or every other day to obtain mid-normal serum testosterone levels. At the typical trans-cis isomer ratio of 62:38, this translates maximally to a daily or every other day trans-isomer dose of only 15.5 mg of trans-isomer; and a cis-isomer dose of 9.5 mg. As indicated above, there has not been any FDA-approved oral therapy for male hypogonadism except methyltestosterone. CLOMID® is administered “off label” to men to treat secondary hypogonadism or male infertility. In some cases, men are prescribed a daily dose of 50 mg of CLOMID®. A daily dose of 50 mg CLOMID® translates at the typical trans-cis isomer ratio of 62:38 to a 31.5 mg of the trans-isomer and 19 mg of the cis-isomer.

U.S. Pat. No. 7,759,360 (Joseph Podolski) disparages the use of CLOMID® for treatment of hypogonadism in men. According to this patent, “[c]lomiphene has also been associated with numerous side effects including: blurred vision, abdominal discomfort, gynecomastia, testicular tumors, vasomotor flushes, nausea, and headaches. Furthermore, other studies suggest that clomiphene possesses both genotoxic and tumor enhancement effects. The net outcome of these observations is that clomiphene in its current format, having between 30% and 50% of the cis isomer, would be unacceptable for chronic therapy in men for the treatment of testosterone deficiency.” See, col. 3, line 63, continuing over to col. 4, line 4.

The remainder of the tenor of the '360 patent is that the cis-isomer should be removed from CLOMID® to improve the drug's efficacy in treating secondary hypogonadism. Baboon data were provided testing CLOMID® against pure trans-isomer (ENCLOMID™) showing “that only ENCLOMID™ significantly and statistically raised total serum testosterone”; col. 6, lines 33-34. Moreover, the cis-isomer of clomiphene actually inhibited LH secretion and reduced peripheral testosterone towards castrate blood levels. These clinical observations were only based on the range of doses that were tested in baboons (S. Hill et al, IDrugs, 12(2): 109-119 (2009))

Moskovic et al., BJU Internat., 110: 1524-1528 (2012), investigated the safety of CLOMID® for long-term management of hypogonadism in men and found it safe. Because cis-clomiphene is an estrogen agonist, some in the art had concerns about the effect of administering CLOMID® over the long-term. The authors found the drug raised T levels substantially in addition to improving osteopenia/osteoporosis. However, the findings here are in direct contrast to a later, wider study by Kacker et al., J. Urol., 191: 1072-1076 (2014), wherein the authors report that CLOMID® actually decrease bone mineral density and increase the risk of fractures at currently available commercial doses (25 mg PO daily or 50 mg PO every other day). Accordingly, the state of the art respecting the long-term safety of using CLOMID® on bone to treat hypogonadism in men remains unclear. We believe the discrepancy may be explained by dose, dose schedule, and ratio of the respective trans- and cis-isomers of clomiphene. Current commercial dose form of CLOMID® is not adequate to bring most men with secondary hypogonadism into the normal T range and side effects include hot flashes and bone loss and osteoporosis. See, Kacker et al., J. Urol., 191: 1072-1076 (2014).

Repros Therapeutics, owner of the '360 patent, is seeking FDA approval to market ANDROXAL® (trans clomiphene citrate) for effectively raising serum testosterone levels in men. ANDROXAL® is purified clomiphene citrate trans isomer containing only trace amounts of cis isomer. Because ANDROXAL® contains little or no estrogenic cis isomer presumably its subjects will not have any side effects due to the estrogenic activity of the cis isomer such as reduction in LH and suppression of endogenous T. Nevertheless, recent clinical data with ANDROXAL® establishes other significant side effects, which the company characterized as being “mild to moderate” in intensity. The side effects included upper respiratory tract infections (11.6%), but the company notes the study was conducted during the cold, flu and allergy season; headache (6.6%); muscle spasms (4%); fatigue (2.8%); and transient blurred vision (1.4%) (changes in visual acuity were noted as an adverse event in only 0.2%). Increase in number of skeletal fractures in ANDROXAL® treated versus placebo subjects was also noted in clinical studies and presented by Repros Therapeutics presumably because of the antiestrogenic effects on bone resulting in bone loss and osteoporosis with acute and chronic use (Repros website clinical data).

Thus, there remains a need for an oral therapy for male secondary hypogonadism that is suitable for long-term therapy if needed, and is effective in raising testosterone concentrations and safe without significant antiestrogen and estrogen side effects including bone loss and osteoporosis, hot flashes, and suppression of endogenous levels of T.

### SUMMARY OF THE INVENTION

These and other objects were met with the present invention, which relates in a first embodiment to a method of treating secondary hypogonadism in a human male patient in need thereof comprising orally administering to said human male patient an effective amount therefor of both (A) trans-clomiphene or a pharmaceutically acceptable salt thereof and (B) cis-clomiphene or a pharmaceutically acceptable salt thereof, wherein (i) the ratio of (A):(B) administered is about 70:30; (ii) the effective amount of (A) is about 12.5 mg per day or about 25 mg per day; and (iii) the effective amount of (B) is less than 15 mg per day.

The present invention relates in a second embodiment to a pharmaceutical oral dosage form comprising both (A) trans-clomiphene or a pharmaceutically acceptable salt thereof and (B) cis-clomiphene or a pharmaceutically acceptable salt thereof, wherein (i) the ratio of (A):(B) in the dosage form is about 70:30; (ii) the amount of (A) in the dosage form is about 12.5 mg or about 25 mg; and (iii) the amount of (B) in the dosage form is less than 15 mg.

The present invention relates in a third embodiment to using the pharmaceutical dosage form of the invention in a method of raising testosterone levels in a human male patient suffering secondary hypogonadism, said method comprising daily or every other day administering to said human male patient the inventive pharmaceutical oral dosage form.

As will be explained in greater detail below, the present invention in all its embodiments has the potential to minimize certain antiestrogenic drug side effects.

The term “about” as used herein means in connection with the ratio of (A):(B) ±1; and in connection with the amounts ±0.5 mg.

### DETAILED DESCRIPTION OF THE INVENTION

Contrary to the teachings of the '360 patent, the correct dose of the cis-isomer is a valuable component in the treatment of secondary hypogonadism and, therefore, should be retained as a significant component of the dose regimen. While not wishing to be bound by theory, we suspect that the trans-isomer is responsible for certain of the symptoms exhibited by CLOMID®, for example, hot flashes and bone loss, and that the presence of the cis-isomer is helpful in reducing or eliminating these side effects. In particular, we suspect that high levels of the trans-isomer contribute to hot flashes and other side effects of CLOMID® and that the daily dose of the trans-isomer may, in conjunction with the presence of a correct amount of the cis-isomer, reduce or eliminate this side effect. We also suspect that the presence of the cis-isomer has beneficial effects that will be especially helpful in the treatment of the male patient, for example, in maintaining and/or increasing bone density and, therefore, prevention of serious bone fractures. We suspect that additionally the presence of the cis-isomer may reduce the muscle spasms and fatigue associated with ANDROXAL®. Furthermore, the correct amount of cis-isomer will also not negatively affect the rise in LH caused by the trans-isomer, such that the testosterone raising effects of the appropriate combination of cis-isomer and trans-isomer will effectively treat secondary hypogonadism while minimizing certain antiestrogenic drug side effects.

Accordingly, the present invention provides trans-isomer for stimulation of testosterone production near the upper limit of efficacy by increasing LH and the present invention provides the correct cis-isomer dose for reduction or elimination of trans-isomer induced antiestrogenic side effects and for the additional benefit of maintaining and/or increasing bone density and prevention of serious fractures.

The combination therapy of trans- and cis-clomiphene is expected to restore normal serum testosterone levels in males suffering secondary hypogonadism. This combination therapy is not expected to prevent increases in LH. This combination therapy is expected to ameliorate symptoms of low testosterone levels and without certain significant antiestrogenic side effects like hot flashes or risk to bone owing to a purely trans-isomer or the wrong doses of the combination of the trans- and cis-clomiphene isomers.

The pharmaceutical dosage forms can be prepared according to methods well known in the art. The pharmaceutical dosage form can be prepared by mixing the two individual cis- and trans-isomers in the specified amounts and then combining with the customary excipients and adjuvants. Alternatively, trans-isomer may be added to the conventional racemic mixture, which is, as noted above, typically a 62:38 ratio of trans:cis isomer, until the 70:30 trans:cis ratio is reached. Methods for preparing the individual isomers are well known in the art; see, for example, U.S. Pat. No. 3,848,030, Examples 31 and 32, the pertinent contents of which are incorporated herein by reference.

According to the present invention, a pharmaceutical dosage form is provided comprising both (A) trans-clomiphene or a pharmaceutically acceptable salt thereof and (B) cis-clomiphene or a pharmaceutically acceptable salt thereof, wherein (i) the ratio of (A):(B) in the dosage form is about 70:30; (ii) the amount of (A) in the dosage form is about 12.5 mg or about 25 mg; and (iii) the amount of (B) in the dosage form is less than 15 mg.

In one preferred embodiment, the pharmaceutical oral dosage form comprises about 12.5 mg of (A) and about 5.3 mg of (B).

In another preferred embodiment, the pharmaceutical oral dosage form comprises about 25 mg of (A) and about 10.7 mg of (B).

In the most preferred embodiment (A) is trans-clomiphene citrate and (B) is cis-clomiphene citrate.

According to the present invention, a human male patient suffering secondary hypogonadism can be successfully treated by orally administering to the human male patient an effective amount therefor of both (A) trans-clomiphene or a pharmaceutically acceptable salt thereof and (B) cis-clomiphene or a pharmaceutically acceptable salt thereof, wherein (i) the ratio of (A):(B) administered is about 70:30; (ii) the effective amount of (A) is about 12.5 mg per day or about 25 mg per day; and (iii) the effective amount of (B) is less than 15 mg per day.

In a preferred embodiment, the male is administered a daily dose of about 12.5 mg of (A) and about 5.3 mg of (B). In an especially preferred embodiment, the daily dose of (A) and (B) is contained in a single tablet comprising both (A) and (B).

In another preferred embodiment, the male is administered a daily dose of about 25 mg of (A) and about 10.7 mg of (B). In an especially preferred embodiment, the daily dose of (A) and (B) is contained in a single tablet comprising both (A) and (B).

In a preferred embodiment, the method is carried out with a pharmaceutical dosage form wherein (A) is trans-clomiphene citrate and/or (B) is cis-clomiphene citrate. In the most preferred embodiment (A) is trans-clomiphene citrate and (B) is cis-clomiphene citrate.

The method is carried out for as long as needed. In some cases, administration of the dosage form for a shorter term of one to a few months will be sufficient to return the patient to normal. In other cases, the secondary hypogonadism is chronic and a longer duration therapy will be required. In a preferred embodiment, the treatment is for chronic secondary hypogonadism, and is carried out for at least one year or for the remainder of the patient's life.

In another preferred embodiment, the inventive dosage form can be used in a method of raising testosterone levels in a human male patient suffering secondary hypogonadism while minimizing antiestrogenic side effects like impaired cognition, hot flashes and preventing bone loss, osteoporosis, and serious fractures, the method comprising daily or every other day administering of the inventive dosage form to said human male patient. In a preferred embodiment, the human male patient suffers chronic secondary hypogonadism, and the administering is carried out for at least one year or for the remainder of the patient's life.

The invention will now be described in greater detail with reference to the following example:

### EXAMPLE

### I. Preparation of Clomiphene Citrate Dosage Forms

### (A) 45 mg Oblong Tablets Containing 25 mg of Trans-Clomiphene Citrate Isomer and 10.7 mg of the Cis-Clomiphene Isomer

25 mg of trans-clomiphene citrate and 10.7 mg of cis-clomiphene citrate are provided separately and then mixed with customary amounts of corn starch, lactose, magnesium stearate, pregelatinized corn starch and sucrose to form a mixture and the mixture is pressed on a tablet press into an oblong tablet having a total weight of 45 mg.

### (B) 25 mg Round Tablets Containing 12.5 mg of Trans-Clomiphene Citrate and 5.3 mg of Cis-Clomiphene

In a like manner, 12.5 mg of trans-clomiphene citrate and 5.3 mg of cis-clomiphene citrate are provided separately and then mixed with customary amounts of corn starch, lactose, magnesium stearate, pregelatinized corn starch and sucrose to form a mixture and the mixture is pressed on a tablet press into a round tablet having a total weight of 25 mg.

1. A method of treating secondary hypogonadism in a human male patient in need thereof comprising orally administering to said human male patient an effective amount therefor of both (A) trans-clomiphene or a pharmaceutically acceptable salt thereof and (B) cis-clomiphene or a pharmaceutically acceptable salt thereof, wherein (i) the ratio of (A):(B) administered is about 70:30; (ii) the effective amount of (A) is about 12.5 mg per day or about 25 mg per day; and (iii) the effective amount of (B) is less than 15 mg per day.

2. The method according to claim 1, wherein the male is administered a daily dose of about 12.5 mg of (A) and about 5.3 mg of (B).

3. The method according to claim 2, wherein the daily dose of (A) and (B) is contained in a single tablet comprising both (A) and (B).

4. The method according to claim 1, wherein the male is administered a daily dose of about 25 mg of (A) and about 10.7 mg of (B).

5. The method according to claim 4, wherein the daily dose of (A) and (B) is contained in a single tablet comprising both (A) and (B).

6. The method according to claim 1, wherein (A) is trans-clomiphene citrate and/or (B) is cis-clomiphene citrate.

7. The method according to claim 1, which is for treatment of chronic secondary hypogonadism, and is carried out for at least 6-12 months or for the remainder of the patient's life.

8. A pharmaceutical oral dosage form comprising both (A) trans-clomiphene or a pharmaceutically acceptable salt thereof and (B) cis-clomiphene or a pharmaceutically acceptable salt thereof, wherein (i) the ratio of (A):(B) in the dosage form is about 70:30; (ii) the amount of (A) in the dosage form is about 12.5 mg or about 25 mg; and (iii) the amount of (B) in the dosage form is less than 15 mg.

9. The pharmaceutical oral dosage form according to claim 8, which comprises about 12.5 mg of (A) and about 5.3 mg of (B).

10. The pharmaceutical oral dosage form according to claim 8, which comprises about 25 mg of (A) and about 10.7 mg of (B).

11. A method of raising testosterone levels in a human male patient suffering secondary hypogonadism, said method comprising daily or every other day administering to said human male patient a pharmaceutical oral dosage form according to claim 8.

12. The method according to claim 11, wherein the human male patient suffers chronic secondary hypogonadism, and said administering is carried out for at least 6-12 months or for the remainder of the patient's life.
